(19) United States
(12) Patent Application Publication
(10) Pub. No.: US 2009/0068133 A1
(43) Pub. Date: Mar. 12, 2009

(54) Composition containing a liquid fatty phase gelled with a semi-crystalline copolymer

(76) Inventors: Jean Mondet, Aulnay Sous Bois (FR); Xavier Blin, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/629,704

(22) PCT Filed: May 11, 2005
(86) PCT No.: PCT/EP05/05801

§ 371 Date: Sep. 17, 2007

Related U.S. Application Data
(60) Provisional application No. 60/581,374, filed on Jun. 22, 2004.

(30) Foreign Application Priority Data
Jun. 17, 2004 (FR) 0451187

(51) Int. Cl. A61K 8/72; A61K 8/81; A61Q 1/06; A61Q 1/08; A61Q 1/10; A61Q 15/00; C08F 218/02

(52) U.S. Cl. 424/64; 424/78.03; 424/63; 424/70.7; 424/65; 526/319

(57) ABSTRACT

The invention relates to a semi-crystalline copolymer of organic structure with a melting point of greater than or equal to 30° C. obtained from at least two monomers A and B, A being a lipophilic monomer bearing crystallizable side chain(s) and B being an amorphous lipophilic monomer that is copolymerizable with A, characterized in that the units derived from the monomers A represent less than 50% by weight of the said copolymer. The invention also relates to cosmetic compositions comprising such a copolymer. This composition is in particular in the form of a soft paste that gives a glossy film on keratin materials, especially the lips.

The present invention relates to a composition, especially a cosmetic composition, containing a liquid fatty phase and at least one particular semi-crystalline copolymer. This semi-crystalline copolymer is advantageously soluble in one of the oils of the liquid fatty phase, and allows the viscosity of a composition comprising such a liquid fatty phase to be readily modified. The said composition may be in the form of a low-viscosity lotion, a cream, a paste, a gel, a cast solid or a stick.

Cosmetic compositions whose fatty phase is structured with semi-crystalline polymers with a melting point of greater than or equal to 30° C. have been described in patent applications FR 2 824 264, WO 03/061612, WO 03/061613, FR 2 846 553, WO 04/041220 and WO 04/041150.

The structured compositions described in these documents are solid compositions. The semi-crystalline polymers they contain may be liposoluble or lipodispersible block (diblock, triblock or multiblock) copolymers containing at least one crystallizable block and at least one amorphous block of different chemical nature from the crystallizable chain.

The semi-crystalline polymers they contain may also be semi-crystalline homopolymers or copolymers containing crystallizable side chains with from 50% to 100% by weight of units resulting from the polymerization of one or more lipophilic monomers bearing crystallizable side chain(s). These semi-crystalline polymers are more particularly described in documents U.S. Pat. No. 5,156,911 and WO-A-01/19333. The oils of the described compositions are efficiently rigidified with stearyl and behenyl homopolymers, but it is not possible to gel them or thicken them. In addition, the solid compositions obtained, such as sticks, are matt.

To solve these two major drawbacks, the inventors have discovered that it is possible to use copolymers as defined hereinbelow, the content of monomers containing crystallizable side groups possibly being less than 50% by weight, even as low as 10% by weight, depending on the level of thickening or gelation that it is desired to obtain.

The inventors have discovered, firstly, that semi-crystalline copolymers containing crystallizable side chains with less than 50% by weight of units resulting from the polymerization of one or more monomers bearing crystallizable side chain(s) can satisfactorily gel or thicken a composition comprising a liquid fatty phase.

Prior-art documents describe cosmetic compositions containing a polystearyl (meth)acrylate, a polybehenyl (meth)acrylate or a copolymer thereof with a low proportion of hydrophilic comonomers.

The semi-crystalline copolymers that are the subject of the invention are advantageously obtained by polymerization of one or more monomers bearing crystallizable side chain(s) and of at least one amorphous lipophilic monomer.

The copolymers that are the subject of the invention can appreciably enlarge the types of formulation and of galenical form both for anhydrous oil media and for oil-in-water or water-in-oil emulsions. Compositions of variable thickness such as lotions, milks, creams, pastes, glosses, or cast solids, including sticks, may be readily prepared.

The invention applies in particular to make-up products for the lips, but also for the eyes, for instance eyeliners, in particular in pencil form, and mascaras, especially in cake form, or for the skin, for instance foundations.

More specifically, one subject of the invention is a cosmetic composition comprising at least one semi-crystalline copolymer of organic structure with a melting point of greater than or equal to 30° C., obtained from at least two monomers A and B, A being a lipophilic monomer bearing crystallizable side chain(s) and B being a monomer that is copolymerizable with A, the said copolymer being such that the units derived from the monomers A preferably represent less than 50% by weight of the said copolymer, preferably less than 40%, preferably less than 30%, preferably less than 20% and more preferably less than 10% by weight of the said copolymer.

A subject of the invention is also a semi-crystalline copolymer of organic structure with a melting point of greater than or equal to 30° C. obtained from at least two monomers A and B, A being a lipophilic monomer bearing crystallizable side chain(s) and B being an amorphous lipophilic monomer that is copolymerizable with A.

The melting point of the semi-crystalline copolymer is preferably less than 150° C.

For the purpose of the invention, the term “copolymers” means compounds comprising at least two repeating units, preferably at least three repeating units and more especially at least ten repeating units.

For the purpose of the invention, the term “semi-crystalline copolymer” means a polymer comprising a crystallizable portion consisting of crystallizable side chains and an amorphous portion consisting of the skeleton and another portion of the side chains.

The terms “organic compound” and “of organic structure” mean compounds containing carbon atoms and hydrogen atoms and possibly hetero atoms, for instance S, O, N or P, alone or in combination.

For the purpose of the invention, a “chain” is a sequence of atoms covalently bonded to the polymer skeleton, the said sequence being a pendent or side sequence relative to the polymer skeleton.

The term “crystallizable or crystalline” means a compound that passes reversibly from the crystalline state to the liquid state, depending on whether it is above or below its melting point.

The term “lipophilic monomer” means a monomer whose corresponding homopolymer is soluble to at least 1% by weight in the oil or in at least one of the oils of the liquid fatty phase of the composition of the invention, at a temperature above the melting point or the glass transition temperature of the said homopolymer.

The term “amorphous monomer” means a monomer whose corresponding homopolymer is not crystalline.

The semi-crystalline copolymer advantageously structures the liquid fatty phase of the composition. In particular, the semi-crystalline copolymer thickens, gels or rigidities the said liquid fatty phase.

The semi-crystalline copolymer is advantageously soluble to at least 1% in the oil or in at least one of the oils of the liquid fatty phase, at a temperature above the melting point of the said copolymer.

The semi-crystalline copolymer of organic structure is obtained by copolymerization of at least two monomers A and B, A being a lipophilic monomer bearing crystallizable side chain(s) and B being an amorphous lipophilic monomer that is copolymerizable with A.

The monomer A containing crystallizable chain(s) may correspond to formula X:

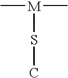

with M representing an atom of the polymer skeleton,
S representing a spacer and
C representing a crystallizable group
   and mixtures thereof, with “S—C” representing an alkyl chain containing at least 11 carbon atoms, which is optionally fluorinated or perfluorinated.

The alkyl chain advantageously contains from 11 to 40 carbon atoms. The monomer A may be a monomer containing vinylic unsaturation bearing an optionally fluorinated hydrocarbon-based crystallizable group containing from 11 to 40 carbon atoms.

The monomer containing a crystallizable chain A may be chosen from saturated C₁₄-C₂₄ alkyl (meth)acrylates, C₁₁-C₁₅ perfluoroalkyl (meth)acrylates, C₁₄ to C₂₄ N-alkyl(meth)acrylamides with or without a fluorine atom, vinyl esters containing C₁₄ to C₂₄ alkyl or perfluoroalkyl chains, vinyl ethers containing C₁₄ to C₂₄ alkyl or perfluoroalkyl chains, C₁₄ to C₂₄ α-olefins, and para-alkylstyrenes with an alkyl group containing from 12 to 24 carbon atoms, and mixtures thereof.

According to one embodiment, the monomer A contains from 11 to 40 carbon atoms, at least six of which are fluorinated.

The monomer B is advantageously a lipophilic amorphous monomer containing vinylic unsaturation, which is preferably non-ionizable. The monomer B may be a monomer bearing at least one C4-50 and preferably C8-30 amorphous alkyl group, which may be linear, branched or cyclic, saturated or unsaturated, and may bear hetero atoms such as O, N, S, P or Si. The hydrogen atoms of the alkyl group may be partially substituted with halogen atoms.

The monomer B preferably comprises at least one branched alkyl group containing between 4 and 50 and preferably from 8 to 30 carbon atoms. The branched alkyl group of the monomer B is chosen, for example, from isobutyl, tert-butyl, isopentyl, tert-hexyl, 2-ethylhexyl, tert-octyl, isononyl, isodecyl, isododecyl, neodecanoyl, isostearyl and octyldodecyl groups, the methyltetradecyl or methylpentadecyl isomers, the methylhexadecyl isomers, the dimethyltetradecyl isomers and the dimethylpentadecyl isomers.

The branched alkyl group of the monomer B is preferably chosen from tert-butyl, tert-hexyl, 2-ethylhexyl, tert-octyl, isononyl, isododecyl, neodecanoyl, isostearyl and octyldodecyl groups.

The monomer B may comprise at least one cyclic alkyl group and/or a linear alkyl group.

The cyclic alkyl group of the monomer B may be chosen from saturated or unsaturated cyclic groups optionally bearing one or more linear or branched C1-15 alkyl substituents.

The cyclic alkyl group of the monomer B is chosen, for example, from saturated cyclic or cycloalkyl groups, such as cyclohexyl, isobornyl, norbornyl or adamantyl groups.

The monomer B may comprise an alkyl group chosen from saturated or unsaturated cyclic groups, such as C4-15 alkyl benzoate groups.

The monomer B is preferably chosen from:

alkyl or cycloalkyl (meth)acrylates,

N-alkyl(meth)acrylamides, N,N-dialkyl(meth)acrylamides and cycloalkyl(meth)acrylamides,

alkyl vinyl esters and cycloalkyl vinyl esters,

alkyl vinyl ethers and cycloalkyl vinyl ethers,

alkyl allyl ethers and cycloalkyl allyl ethers,

α-olefins containing an alkyl or cycloalkyl group,

monoalkyl or dialkyl esters and cycloalkyl esters, in particular maleic acid or itaconic acid esters,

maleic acid or itaconic acid amides, and

mixtures thereof.

The N,N-dialkyl group is advantageously such that at least one of the alkyl groups is of C4-11, the other possibly being identical or different and containing from 1 to 11 carbon atoms.

According to another embodiment, the monomer B comprises at least one linear alkyl group containing between 4 and 11 and preferably between 8 and 11 carbon atoms, such as n-butyl, n-pentyl, n-hexyl, n-heptyl, n-octyl, n-nonyl, n-decyl or n-undecyl, and mixtures thereof.

The proportion of the units resulting from the polymerization of the monomer A in the copolymer may be between 10% and 90% and preferably between 25% and 75%. The proportion of units resulting from the polymerization of the monomer B in the copolymer may be between 10% and 90% and preferably between 25% and 75%.

The semi-crystalline copolymer may have a weight-average molecular mass ranging from 5000 to 1000 000, preferably from 10 000 to 800 000 and preferentially from 15 000 to 500 000. Its melting point is preferably higher than the temperature of the keratinous support intended to receive the said composition.

The semi-crystalline copolymer generally represents from 0.1% to 80% of the total weight of the composition, better still from 0.5% to 40%, even better still from 3% to 30% and preferably from 15% to 25% by weight of the composition.

The units of the semi-crystalline copolymer resulting from the polymerization of the monomer A and of the monomer B may be distributed: randomly (random copolymer), alternately, with a distribution gradient, or in the form of blocks (diblock, triblock, multiblock or pseudo-block copolymers), or in the form of grafts (for at least one from among A and B).

The semi-crystalline copolymers may also result from the copolymerization of the monomer A, of the monomer B and of at least one other monomer that may be hydrophilic, lipophilic or of intermediate polarity.

This other monomer may be, for example, a polar or non-polar monomer or a mixture of the two.

Polar monomers that may be mentioned include a monomer bearing polyoxyalkylene (especially oxyethylene and/or oxypropylene) groups, a hydroxyalkyl (meth)acrylate, for instance hydroxyethyl acrylate, (meth)acrylamide, an N-alkyl(meth)acrylamide, an N,N-dialkyl(meth)acrylamide, for instance N,N-diisopropyl-acrylamide or N-vinylpyrrolidone (NVP), N-vinylcaprolactam, a monomer bearing at least one carboxylic acid group, for instance (meth)acrylic acids, crotonic acid, itaconic acid, maleic acid or fumaric acid, or bearing a carboxylic acid anhydride group, for instance maleic anhydride, and mixtures thereof.

Non-polar monomers that may be mentioned include an ester of the linear or branched C1 to C3 alkyl (meth)acrylate type, a vinyl ester such that the ester group is of C1 to C3, an alkyl vinyl ether such that the alkyl group is of C1 to C3, an α-olefin, styrene or styrene substituted with a C1 to C3 alkyl group, for instance α-methylstyrene, or a macromonomer of the polyorganosiloxane type containing vinylic unsaturation.

The semi-crystalline copolymers of the invention may be partially crosslinked, but, in this case, must remain soluble in the oil phase above their melting point.

The semi-crystalline copolymers of the invention may result from any technique of polymerization of double bonds that is suitable for this type of reaction: free-radical polymerization, controlled free-radical polymerization, ionic polymerization or group-transfer polymerization.

### Mixture of Semi-Crystalline Copolymer and of High-Melting Crystalline Compound

In the description hereinbelow, a “high-melting crystalline compound” is a crystalline or semi-crystalline compound having a melting point m.p.₁ higher than the melting point m.p.₂ of a semi-crystalline copolymer defined previously. According to the invention, the melting point may be measured especially by any known method and in particular using a differential scanning calorimeter (DSC).

The composition advantageously contains a mixture i) of a high-melting crystalline or semi-crystalline compound with a melting point at least equal to 50° C. and ii) of a semi-crystalline copolymer as defined previously with a melting point of less than 50° C.

According to the invention, the high-melting crystalline compound(s) is (are) advantageously compounds with a melting point m.p.₁ such that 50° C.≦m.p.₂≦150° C., better still 55° C.≦m.p.₁≦150° C. and preferably 60° C.≦m.p.₁≦130° C.

The semi-crystalline copolymers advantageously have a melting point m.p.₂ such that 30° C.≦m.p.₂≦50° C. and better still 35° C.≦m.p.₂≦45° C. This melting point is a first-order change of state temperature.

As high-melting compound that may be used in the invention, mention may be made of high-melting waxes, for instance certain polyethylene waxes such as Epolene N-14 sold by Eastman Chemical Co., carnauba waxes and certain microcrystalline waxes, for instance those sold by Tisco under the brand name “Tisco Wax 88”.

The high-melting compound may be a semi-crystalline copolymer as described previously, with a melting point of greater than or equal to 50° C. In this embodiment, the low-melting semi-crystalline copolymer and the high-melting copolymer may or may not be obtained from the same monomers A and B.

The high-melting compound may be a semi-crystalline polymer.

The high-melting semi-crystalline polymer in the composition of the invention advantageously has a weight-average molecular mass Mw ranging from 5000 to 1 000 000, preferably from 10 000 to 800 000 and preferentially from 15 000 to 500 000.

The high-melting semi-crystalline polymer according to the invention can structure the liquid fatty phase.

According to the invention, the high-melting semi-crystalline polymer is advantageously soluble in the fatty phase, especially to at least 1% by weight, at a temperature above its melting point.

Preferably, the semi-crystalline polymer is obtained from at least one monomer containing a crystallizable side chain, or the semi-crystalline polymer comprises a crystallizable block.

The said polymer is a homopolymer or a copolymer.

The high-melting semi-crystalline polymers of the invention containing crystallizable blocks may be block or multiblock copolymers. They may be obtained by polymerizing a monomer containing reactive (or ethylenic) double bonds or by polycondensation. When the high-melting polymers of the invention are polymers containing crystallizable side chains, these side chains are advantageously in random or statistical form.

Preferably, the high-melting semi-crystalline polymers of the invention are of synthetic origin.

The high-melting semi-crystalline polymers that may be used in the invention are in particular:

block copolymers of polyolefins of controlled crystallization, whose monomers are described in EP-A-0 951 897,

polycondensates, especially of aliphatic or aromatic polyester type or of aliphatic/aromatic polyester type,

homopolymers or copolymers bearing at least one crystallizable side chain and homopolymers or copolymers bearing in the skeleton at least one crystallizable block, for instance those described in document U.S. Pat. No. 5,156,911,

homopolymers or copolymers bearing at least one crystallizable side chain, in particular bearing fluoro group(s), such as those described in document WO-A-01/19333,

and mixtures thereof.

### A) High-Melting Semi-Crystalline Polymers Containing Crystallizable Side Chains

Mention may be made in particular of those defined in documents U.S. Pat. No. 5,156,911 and WO-A-01/19333.

Mention may be made of homopolymers or copolymers comprising from 50% to 100% by weight of units resulting from the polymerization of one or more lipophilic monomers bearing a crystallizable side chain.

Mention may be made of semi-crystalline copolymers as described previously, the melting point of which is greater than or equal to 50° C., i.e. copolymers resulting from the copolymerization between at least one lipophilic and crystallizable monomer A and at least one lipophilic and amorphous monomer B in a proportion representing from 10% to 90% by weight of the polymer, the proportion of the monomer A being between 10% and 90% of the total weight of the monomers reacted together.

These homopolymers or copolymers are of any nature, provided that they meet the conditions mentioned hereinbelow with, in particular, the characteristic of being soluble or dispersible in the liquid fatty phase, by heating above their melting point m.p. They can result:

from the polymerization, especially the free-radical polymerization, of one or more monomers containing reactive or ethylenic double bond(s) with respect to a polymerization, namely a vinyl, (meth)acrylic or allylic group,

from the polycondensation of one or more monomers bearing co-reactive groups (carboxylic acid, sulfonic acid, alcohol, amine or isocyanate), such as, for example, polyesters, polyurethanes, polyethers, polyureas or polyamides.

a) In general, the crystallizable units (chains or blocks) or semi-crystalline polymers according to the invention are derived from monomer(s) containing crystallizable block(s) or chain(s), used for manufacturing semi-crystalline polymers. These polymers are chosen especially from homopolymers and copolymers resulting from the polymerization of at least one monomer A containing crystallizable chain(s) that may be represented by formula X:

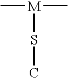

with M representing an atom of the polymer skeleton,
S representing a spacer and
C representing a crystallizable group

The crystallizable chains “—S—C” may be aliphatic or aromatic, and optionally fluorinated or perfluorinated. “S” especially represents a group (CH₂)ₙ, or (CH₂CH₂O)ₙ or (CH₂O), which may be linear or branched or cyclic, with n being an integer ranging from 0 to 22. Preferably, “S” is a linear group. Preferably, “S” and “C” are different.

When the crystallizable chains are hydrocarbon-based aliphatic chains, they comprise hydrocarbon-based alkyl chains containing at least 11 carbon atoms and not more than 40 carbon atoms and better still not more than 24 carbon atoms. They are especially aliphatic chains or alkyl chains containing at least 12 carbon atoms, and they are preferably C₁₄-C₂₄ alkyl chains, preferably C₁₆-C₂₂ alkyl chains. When they are fluoroalkyl or perfluoroalkyl chains, they contain at least 11 carbon atoms, at least six of which carbon atoms are fluorinated.

As examples of high-melting semi-crystalline homopolymers or copolymers containing crystallizable chain(s), mention may be made of those resulting from the polymerization of one or more of the following monomers: (meth)acrylates of saturated alkyls with the alkyl group being C₁₄-C₂₄, perfluoroalkyl (meth)acrylates with a C₁₁-C₁₅ perfluoroalkyl group, N-alkyl(meth)acrylamides with the alkyl group being C₁₄ to C₂₄ with or without a fluorine atom, vinyl esters containing alkyl or perfluoro(alkyl) chains with the alkyl group being C₁₄ to C₂₄ (with at least six fluorine atoms per perfluoroalkyl chain), vinyl ethers containing alkyl or perfluoro(alkyl) chains with the alkyl group being C₁₄ to C₂₄ and at least six fluorine atoms per perfluoroalkyl chain, C₁₄ to C₂₄ α-olefins such as, for example, octadecene, para-alkylstyrenes with an alkyl group containing from 12 to 24 carbon atoms, and mixtures thereof.

When the high-melting polymers result from a polycondensation, the hydrocarbon-based and/or fluorinated crystallizable chains as defined above are borne by a monomer that may be a diacid, a diol, a diamine or a diisocyanate.

When the high-melting polymers of the invention are copolymers, they additionally contain from 0 to 50% of groups Y or Z resulting from the copolymerization:

α) of Y which is a polar or non-polar monomer or a mixture of the two:

When Y is a polar monomer, it is either a monomer bearing polyoxyalkylenated groups (especially oxyethylenated and/or oxypropylenated groups), a hydroxyalkyl (meth)acrylate, for instance hydroxyethyl acrylate, (meth)acrylamide, an N-alkyl(meth)acrylamide, an N,N-dialkyl(meth)acrylamide such as, for example, N,N-diisopropylacrylamide or N-vinylpyrrolidone (NVP), N-vinylcaprolactam, a monomer bearing at least one carboxylic acid group, for instance (meth)acrylic acid, crotonic acid, itaconic acid, maleic acid or fumaric acid, or bearing a carboxylic acid anhydride group, for instance maleic anhydride, and mixtures thereof.

When Y is a non-polar, amorphous and preferably lipophilic monomer, it may preferably be identical to B as defined for the low-melting polymer, in particular: an ester of the linear, branched or cyclic alkyl (meth)acrylate type, a vinyl ester, an alkyl vinyl ether, or alternatively: an α-olefin, styrene or styrene substituted with a C₁ to C₁₀ alkyl group, for instance α-methylstyrene, or a macromonomer of the polyorganosiloxane type containing vinyl unsaturation.

For the purposes of the invention, the term “alkyl” means a saturated group especially of C₈ to C₂₄, except where otherwise mentioned.

Preferably, a semi-crystalline copolymer resulting from the copolymerization between at least one crystalline monomer A and at least one lipophilic and amorphous monomer B will preferably be used as high-melting semi-crystalline polymer, the units derived from the monomers A representing less than 50% by weight relative to the total weight of the said copolymer.

β) of Z which is a polar monomer or a mixture of polar monomers. In this case, Z has the same definition as the “polar Y” defined above.

### B) High-Melting Polymers Bearing in the Skeleton at Least One Crystallizable Block

This is also a case of polymers that are soluble or dispersible in the liquid fatty phase by heating above their melting point m.p. These polymers are especially block copolymers consisting of at least two blocks of different chemical nature, one of which is crystallizable.

The polymers defined in U.S. Pat. No. 5,156,911 may be used;

Block copolymers of olefin or of cycloolefin containing a crystallizable chain, for instance those derived from the block polymerization of:

cyclobutene, cyclohexene, cyclooctene, norbornene (i.e. bicyclo(2,2,1)-2-heptene), 5-methylnorbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinylnorbornene, 1,4,5,8-dimethano-1,2,3,4,4a,5,8a-octahydronaphthalene, dicyclopentadiene, or mixtures thereof,

with ethylene, propylene, 1-butene, 3-methyl-1-butene, 1-hexene, 4-methyl-1-pentene, 1-octene, 1-decene or 1-eicosene, or mixtures thereof,

and in particular copoly(ethylene/norbornene) blocks and (ethylene/propylene/ethylidene-norbornene) block terpolymers. Those resulting from the block copolymerization of at least 2 C₂-C₁₆, better still C₂-C₁₂ α-olefins such as those mentioned above and in particular block bipolymers of ethylene and of 1-octene may also be used.

Crystalline polymers that are solid at room temperature and that have a melting point of greater than 50° C. or random polymers comprising a controlled crystallization, as described in document EP-A-0 951 897 and more especially the commercial products Engage 8 401 and Engage 8 402 from Dupont de Nemours, respectively having melting points of 51° C. and 64° C. and which are ethylene/1-octene random bipolymers, may be used as high-melting crystalline compound.

The copolymers may be copolymers containing at least one crystallizable block, the copolymer residue being amorphous (at room temperature). These copolymers may also contain two crystallizable blocks of different chemical nature. The preferred copolymers are those that simultaneously contain at room temperature a crystallizable block and an amorphous block that are both hydrophobic and lipophilic, sequentially distributed; mention may be made, for example, of polymers containing one of the crystallizable blocks and one of the amorphous blocks below:

Block that is crystallizable by nature: a) polyester, for instance poly(alkylene terephthalate), b) polyolefin, for instance polyethylenes or polypropylenes.

Amorphous and lipophilic block, for instance: amorphous polyolefins or copoly(olefin)s such as poly(isobutylene), hydrogenated polybutadiene or hydrogenated poly(isoprene).

As examples of such high-melting copolymers containing a crystallizable block and an amorphous block, mention may be made of:

α) poly(ε-caprolactone)-b-poly(butadiene) block copolymers, preferably used hydrogenated, such as those described in the article D6 “Melting behavior of poly(-caprolactone)-block-polybutadiene copolymers” from S. Nojima, Macromolecules, 32, 3727-3734 (1999),
β) the hydrogenated block or multiblock poly(butylene terephthalate)-b-poly(isoprene) block copolymers cited in the article D7 “Study of morphological and mechanical properties of PP/PBT” by B. Boutevin et al., Polymer Bulletin, 34, 117-123 (1995),
γ) the poly(ethylene)-b-copoly(ethylene/propylene) block copolymers cited in the articles D8 “Morphology of semi-crystalline block copolymers of ethylene-(ethylene-alt-propylene)” by P. Rangarajan et al., Macromolecules, 26, 4640-4645 (1993) and D9 “Polymer aggregates with crystalline cores: the system poly(ethylene)-poly(ethylene-propylene)” by P. Richter et al., Macromolecules, 30, 1053-1068 (1997),
δ) the poly(ethylene)-b-poly(ethylethylene) block copolymers cited in the general article D10 “Crystallization in block copolymers” by I. W. Hamley, Advances in Polymer Science, Vol. 148, 113-137 (1999).

The high-melting semi-crystalline polymers in the composition of the invention may or may not be partially crosslinked, provided that the degree of crosslinking does not interfere with their dissolution or dispersion in the liquid fatty phase by heating above their melting point. It may then be a chemical crosslinking, by reaction with a multifunctional monomer during the polymerization. It may also be a physical crosslinking which may, in this case, be due either to the establishment of bonds of hydrogen or dipolar type between groups borne by the polymer, such as, for example, the dipolar interactions between carboxylate ionomers, these interactions being of small amount and borne by the polymer skeleton; or to a phase separation between the crystallizable blocks and the amorphous blocks borne by the polymer.

Preferably, the high-melting semi-crystalline polymers do not comprise any carboxylic groups.

According to the invention, the high-melting compound (crystalline or semi-crystalline) and the semi-crystalline copolymer are advantageously in a weight ratio ranging from 10/90 to 90/10, better still from 40/60 to 60/40 and more preferably in a weight ratio of close to 50/50.

Preferably, the high-melting semi-crystalline polymers in the composition according to the invention are non-crosslinked.

As specific examples of the high-melting semi-crystalline polymer that may be used in the composition according to the invention, mention may be made of the products Intelimer from the company Landec, described in the brochure “Intelimer polymers”, Landec IP22 (Rev. 4-97). These polymers are in solid form at room temperature (25° C.). They bear crystallizable side chains and have the formula X above.

### Liquid Fatty Phase

For the purposes of the patent application, the term “liquid fatty phase” means a fatty phase that is liquid at room temperature (25° C.) and atmospheric pressure (760 mmHg), composed of one or more fatty substances that are liquid at room temperature, also known as oils.

The liquid fatty phase may contain one or more apolar oils, a mixture of apolar oil(s) and of polar oil(s), or a mixture of polar oils.

In particular, the “apolar oils” have a solubility parameter δₐ=0.

The term “polar oil” means an oil composed of chemical compounds comprising at least one polar group. The “polar groups” are well known to those skilled in the art; they may be, for example, ionic polar groups or nonionic groups chosen from —COOH; —OH; ethylene oxide; propylene oxide; —PO₄; —NHR; —NR₁R₂ with R₁ and R₂ optionally forming a ring and representing a linear or branched C₁ to C₂₀ alkyl or alkoxy radical.

The sparingly polar oils comprise oils that have a mean solubility parameter at 25° C. of:

0<δₐ<5.0 (J/cm³)^{1/2}.

The highly polar oils have a mean solubility parameter δₐ according to the Hansen solubility space, at 25° C., of: δₐ≧5.0 (J/cm³)^{1/2}.

The definition and calculation of the solubility parameters in the three-dimensional Hansen solubility space are described in the article by C. M. Hansen: “The three dimensional solubility parameters” J. Paint Technol. 39, 105 (1967).

According to this Hansen space:

δ_{D} characterizes the London dispersion forces derived from the formation of dipoles induced during molecular impacts;

δₚ characterizes the Debye interaction forces between permanent dipoles and the Keesom interaction forces between induced dipoles and permanent dipoles;

δₕ characterizes the specific interaction forces (such as hydrogen bonding, acid/base, donor/acceptor, etc.),

δₐ is determined by the equation: δₐ=(δₚ²+δₕ²)^{1/2}.

The parameters δₚ, δₕ, δ_{D} and δₐ are expressed in (J/cm³)^{1/2}.

When the liquid fatty phase is a mixture of different oils, the solubility parameters of the mixture are determined from those of the compounds taken separately, according to the following relationships:

$${δ}_{Dmixt} = {\sum }_{i} xi {δ}_{Di} ; {δ}_{pmixt} = {\sum }_{i} xi {δ}_{pi} and {δ}_{hmixt} = {\sum }_{i} xi {δ}_{hi}$$

$${δ}_{amixt} = {( {δ}_{pmixt}^{2} + {δ}_{hmixt}^{2} )}^{1 / 2}$$

in which xi represents the volume fraction of compound i in the mixture.

The liquid fatty phase may contain at least one volatile oil.

The term “volatile oil” means any oil with a vapour pressure, at room temperature and atmospheric pressure, of greater than 0.13 Pa.

The volatile oil(s), which is (are) liquid at room temperature, especially has (have) a vapour pressure, measured at room temperature and atmospheric pressure, ranging from 0.13 Pa to 40 000 Pa, preferably from 0.26 Pa to 40 000 Pa and better still ranging from 1.3 Pa to 1300 Pa.

According to the invention, these volatile oils especially facilitate the application of the composition to the skin, the lips or the integuments. These oils may be hydrocarbon-based oils, silicone oils optionally comprising alkyl or alkoxy groups that are pendent or at the end of a silicone chain, or a mixture of these oils.

Preferably, the volatile oils are chosen from hydrocarbon-based oils of animal or plant origin and silicone oils, and mixtures thereof. Preferably, the volatile oils are cosmetic oils chosen from oils that do not have a flashpoint, oils with a flashpoint ranging from 40° C. to 100° C., and mixtures thereof, in order to facilitate their use. In addition, they advantageously have a boiling point at atmospheric pressure of less than 220° C. and better still less than 210° C., especially ranging from 110 to 210° C.

As volatile oils that may be used in the invention, mention may be made of linear or cyclic silicone oils with especially a viscosity at room temperature of less than 5 cSt and especially containing from 2 to 10 silicon atoms, preferably from 2 to 7 silicon atoms, these silicones optionally comprising alkyl or alkoxy groups containing from 1 to 10 carbon atoms.

As volatile silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

As other volatile oils that may be used in the invention, mention may be made of hydrocarbon-based volatile oils containing from 8 to 16 carbon atoms, and mixtures thereof, and especially branched C₈-C₁₆ alkanes, for instance C₈-C₁₆ isoalkanes (also known as isoparaffins), isododecane, isodecane and isohexadecane, and for example the oils sold under the trade names Isopars or Permethyls, and mixtures thereof.

Preferably, isododecane (Permethyls 99 A), C₈-C₁₆ isoparaffins, for instance Isopar L, E, G or H, and mixtures thereof, optionally combined with decamethyltetrasiloxane or with cyclopentasiloxane, are used.

Volatile fluoro oils may also be used.

These volatile oils especially represent from 0.1% to 98% of the total weight of the composition, better still from 2% to 75% and preferably between 5% and 50% of the total weight of the composition. This amount will be adapted by a person skilled in the art depending on the intensity of the desired properties.

The weight ratio of volatile oil relative to the semi-crystalline copolymer is advantageously between 0.5 and 2.5 and preferably from 1 to 2.

The liquid fatty phase may contain a monoester oil, which preferably contains from 10 to 40 carbon atoms, preferably from 10 to 30 carbon atoms, more preferably from 12 to 26 carbon atoms and more preferably from 16 to 22 carbon atoms.

The ester according to the invention is preferably liquid at room temperature (25° C.). It is advantageously aliphatic and/or linear.

The ester preferably represents from 1% to 40%, better still from 5% to 30% and preferably from 20% to 25% by weight of the composition.

The weight ratio of the ester relative to the semi-crystalline copolymer is advantageously between 1 and 2 and preferably between 1.3 and 1.7.

The ester according to the invention may be chosen from:
synthetic esters, especially of fatty acids, for instance the oils of formula R₁COOR₂ in which R₁ represents a higher fatty acid residue containing from 1 to 38 carbon atoms and R₂ represents a hydrocarbon-based chain containing from 1 to 38 carbon atoms with 39≧R₁+R₂≧10, for instance purcellin oil, isononyl isononanoate, isopropyl isostearate, isopropyl myristate, 2-ethylhexyl palmitate, 2-octyldodecyl stearate, 2-octyldodecyl erucate or isostearyl isostearate;
hydroxylated esters, for instance isostearyl lactate, octyl hydroxystearate, octyldodecyl hydroxystearate and fatty alcohol heptanoates, octanoates or decanoates;
oleyl erucate;
a C12-C15 alkyl benzoate ester (for instance Finsolv TN sold by the company Finetex);
and mixtures thereof.

The liquid fatty phase may contain at least one apolar oil chosen in particular from silicone oils such as linear or cyclic polydimethylsiloxanes (PDMS), which are liquid at room temperature; polydimethylsiloxanes containing alkyl, alkoxy or phenyl groups, pendent and/or at the end of a silicone chain, these groups containing from 2 to 24 carbon atoms and being liquid at room temperature; phenylsilicones, for instance phenyl trimethicones, phenyl dimethicones, phenyl trimethylsiloxy diphenylsiloxanes, diphenyl dimethicones, diphenyl methyldiphenyl trisiloxanes, and 2-phenylethyl trimethylsiloxysilicates, which are liquid; linear or branched hydrocarbons or fluorocarbons of synthetic or mineral origin, which are liquid, for instance liquid paraffins and derivatives thereof, petroleum jelly, polydecenes, hydrogenated polyisobutene such as Parleam® sold by the company Nippon Oil Fats, and squalane; mixtures thereof. Preferably, the apolar oils used are apolar oils of the liquid hydrocarbon-based type, of mineral or synthetic origin, chosen especially from Parleam® oil (hydrogenated isoparaffin), isoparaffins and squalane, and mixtures thereof.

In particular, the polar oils may be chosen from:

hydrocarbon-based plant oils with a high content of triglycerides consisting of fatty acid esters (of C₄ to C₂₄) of glycerol in which the fatty acids may have varied chain lengths, these chains possibly being linear or branched, and saturated or unsaturated; these oils are, in particular, wheat germ oil, corn oil, sunflower oil, shea butter, castor oil, sweet almond oil, macadamia oil, apricot oil, soybean oil, rapeseed oil, cotton oil, alfalfa oil, poppy oil, pumpkin oil, sesame oil, marrow oil, avocado oil, hazelnut oil, grapeseed oil, blackcurrant seed oil, evening primrose oil, millet oil, barley oil, quinoa oil, olive oil, rye oil, safflower oil, candlenut oil, passionflower oil and musk rose oil; or alternatively caprylic/capric acid triglycerides such as those sold by the company Stearineries Dubois or those sold under the names Miglyol 810, 812 and 818 by the company Dynamit Nobel, and heptanoic acid or octanoic acid triglycerides;

synthetic esters and ethers, for instance isopropyl myristate, 2-ethylhexyl palmitate, alcohol or polyalcohol octanoates, decanoates or ricinoleates, and hydroxylated esters, for instance isostearyl lactate or diisostearyl malate; and pentaerythritol esters;

fatty acids containing from 12 to 22 carbon atoms such as oleic acid, linoleic acid or linolenic acid;

fatty alcohols that are liquid at room temperature, the branched and/or unsaturated carbon-based chain of which contains from 12 to 26 carbon atoms, especially 2-octyldodecanol, isostearyl alcohol, oleyl alcohol, 2-hexyldecanol, 2-butyloctanol and 2-undecylpentadecanol;

mixtures thereof.

The liquid fatty phase represents, in practice, from 5% to 99% and preferably from 20% to 80% of the total weight of the composition. It advantageously represents at least 60% of the total weight of the composition.

### Dyestuff

The composition advantageously contains a dyestuff, which may be chosen from the lipophilic dyes, hydrophilic dyes, pigments and nacres usually used in cosmetic or dermatological compositions, and mixtures thereof. This dyestuff is generally present in a proportion of from 0.01% to 50% (solids) and preferably from 5% to 30% of the total weight of the composition (if present).

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow or methyl yellow. They can represent from 0% to 20% of the weight of the composition and better still from 0.01% to 6% (if present). The water-soluble dyes are, for example, beetroot juice or methylene blue, and can represent up to 6% of the total weight of the composition.

The pigments may be white or coloured, mineral and/or organic, and coated or uncoated. Among the mineral pigments which may be mentioned are titanium dioxide or zinc dioxide, optionally surface-treated, zirconium oxide or cerium oxide, as well as iron oxide, chromium oxide, manganese violet, ultramarine blue, chromium hydrate and ferric blue. Among the organic pigments that may be mentioned are carbon black, pigments of D&C type, and lakes based on cochineal carmine or on barium, strontium, calcium or aluminium (such as D&C Red 27, 21 or 7, D&C Yellow 5 or 6 and FD&C Blue No. 1). The pigments can represent from 0% to 40% (0.01% to 40%), especially from 0.5% to 35% and better still from 2% to 25% of the total weight of the composition (if present).

The nacreous pigments may be chosen from white nacreous pigments such as mica coated with titanium or with bismuth oxychloride, coloured nacreous pigments such as titanium mica with iron oxides, titanium mica with, in particular, ferric blue or chromium oxide, titanium mica with an organic pigment of the type mentioned above, as well as nacreous pigments based on bismuth oxychloride. They can represent from 0 to 25% (0.05% to 25%) and better still from 0.1% to 15% of the total weight of the composition (if present).

Advantageously, the pigments and nacres are introduced into the composition in the form of a pigmentary paste.

For the purposes of the invention, the term “pigmentary paste” means a concentrated colloidal dispersion of coated or uncoated coloured particles in a continuous medium, optionally stabilized using a dispersant.

### Wax

At least one wax different from one of the waxes described previously may also be used in the composition of the invention, in the case of a mixture of the semi-crystalline copolymer with a high-melting crystalline compound.

For the purposes of the present invention, a wax is a lipophilic fatty compound that is solid at room temperature (25° C.) and at atmospheric pressure (760 mm Hg), which undergoes a reversible solid/liquid change of state, having a melting point of greater than 30° C., which may be up to 200° C., and having an anisotropic crystal organization in the solid state.

For the purposes of the invention, the melting point of the wax corresponds to the temperature of the most endothermic peak observed by thermal analysis (DSC) as described in standard ISO 11357-3; 1999.

The melting point of the wax may be measured using a differential scanning calorimeter (DSC), for example the calorimeter sold under the name MDSC 2920 by the company TA Instruments.

The measuring protocol is, for example, as follows:

A sample of 5 mg of wax placed in a crucible is subjected to a first temperature increase ranging from −20° C. to 100° C., at a heating rate of 10° C./minute, and is then cooled from 100° C. to −20° C. at a cooling rate of 10° C./minute and is finally subjected to a second temperature increase ranging from −20° C. to 100° C. at a heating rate of 5° C./minute. During the second temperature increase, the variation of the difference in power absorbed by the empty crucible and by the crucible containing the sample of wax is measured as a function of the temperature. The melting point of the compound is the value of the temperature corresponding to the top of the peak of the curve representing the variation of the difference in power absorbed as a function of the temperature.

The waxes are especially of natural origin, for instance beeswax, candelilla wax, ouricury wax, Japan wax, cork fibre wax or sugarcane wax, paraffin wax, lignite wax, microcrystalline waxes, lanolin wax, ceresin wax, ozokerite wax, montan wax, polyethylene waxes derived from the polymerization of ethylene and the waxes obtained by Fischer-Tropsch synthesis, esters of fatty acids and glycerides, and silicone waxes, for instance alkyl or alkoxy poly(di)methylsiloxanes and/or poly(di)methylsiloxane esters.

Advantageously, the composition of the invention contains little or no “matting” fillers and in particular less than 5% of matting filler. This is especially the case when it is desired to obtain a glossy deposit on keratin materials such as the lips, the eyelashes and the hair. In contrast, fillers of this type may be used for a foundation. A matting filler is generally a filler that absorbs the skin's sweat and/or sebum, for instance silicas, talcs, clays, kaolins and polyamide powders (Nylon®).

The composition may also comprise a pasty compound, i.e. a compound containing a liquid fraction and a solid fraction at 23° C.

Needless to say, a person skilled in the art will take care to select the optional additional additives and/or the amount thereof such that the advantageous properties of the composition according to the invention are not, or are not substantially, adversely affected by the envisaged addition.

The makeup composition of the invention may be in the form of a coloured product, in particular for the skin, optionally having care or treating properties, and may be in particular a foundation, a blusher, a makeup rouge, an eyeshadow, a concealer product, an eyeliner or a body makeup product; a lip makeup product, for instance a lipstick, a lip gloss or a lip pencil optionally having care or treating properties; a makeup product for the integuments, for instance the nails or the eyelashes, in the form of mascara, or for the eyebrows and the hair.

It may be in the form of a water-in-oil or oil-in-water emulsion or in anhydrous form.

The term “anhydrous” refers to a composition comprising less than 5% by weight of water, preferably less than 3% and more preferably less than 1% by weight of water.

A subject of the invention is also a cosmetic process for caring for, making up or treating human keratin materials and especially human skin, facial lips and integuments, comprising the application to the keratin materials of the composition, especially the cosmetic composition, as defined above.

A subject of the invention is also a copolymer as described previously.

A subject of the invention is also the use of a copolymer as described previously, in a cosmetic composition containing at least one liquid fatty phase, in order to gel, thicken or structure the said liquid fatty phase.

The invention is illustrated in greater detail in the examples that follow. The amounts are given as mass percentages.

### I) EXAMPLES OF MANUFACTURING SEMI-CRYSTALLINE COPOLYMERS

### Example 1

### Copoly(Stearyl Acrylate/Isostearyl Acrylate) 75/25 by Weight

120 g of Parleam are introduced into a 1 litre reactor equipped with a central anchor stirrer, a condenser and a thermometer, and are heated from room temperature to 80° C. over 45 minutes. At 80° C., the mixture C1 below is introduced over 2 hours:

40 g of cyclohexane+4 g of Trigonox 141 [2,5-bis(2-ethylhexanoylperoxy)-2,5-dimethylhexane].

30 minutes after the start of addition of the mixture C1, the mixture C2 consisting of:

150 g of stearyl acrylate+50 g of isostearyl acrylate (sold by Prochema)+400 g of cyclohexane
is introduced over 1 hour 30 minutes.

After the two additions, the mixture is left to react for a further three hours at 80° C. All of the cyclohexane present in the reaction medium is then distilled off at atmospheric pressure.

The copolymer is thus obtained at a concentration of 60% active material in the Parleam.

Its weight-average molecular mass is about 20 000-30 000 and its melting point m.p. is 48° C., measured by DSC.

### Example 2

### Copoly(Stearyl Acrylate/Isostearyl Acrylate) 50/50 by Weight

The same procedure as in Example 1 is applied, except that the monomer mixture used consists of:

100 g of stearyl acrylate
100 g of isostearyl acrylate.

The polymer obtained is also at a concentration of 60% by weight of active material in the Parleam, its weight-average molecular mass is 35 000-40 000 and its melting point is 45° C.

### Example 3

### Copoly(Behenyl Acrylate/Isostearyl Acrylate) 75/25 by Weight

The same procedure as in Example 1 is applied, except that the monomer mixture used consists of:

150 g of behenyl acrylate
50 g of isostearyl acrylate.

The copolymer obtained is at a concentration of 60% active material in the Parleam. Its weight-average molecular mass is about 20 000-25 000. Its melting point is 57° C.

### Example 4

### Copoly(Behenyl Acrylate/Isostearyl Acrylate) 50/50 by Weight

The same procedure as in Example 1 is applied, except that the monomer mixture used consists of:

100 g of behenyl acrylate
100 g of isostearyl acrylate.

The copolymer obtained is at a concentration of 60% active material in the Parleam. Its weight-average molecular mass is 25 000-30 000 and its melting point is 55° C.

### II) EXAMPLE OF A LIPSTICK COMPOSITION

The percentages are by weight.

| | |
|---|---|
| Copolymer of Example 1 | 14% |
| Octyldodecanyl | 10% |
| Pentaerythrityl tetraisostearate | 22% |
| Tridceyl trimellitate | 22% |
| Pigments | 4% |
| Diisostearyl malate | qs 100% |

1-44. (canceled)

45. A cosmetic composition comprising a liquid fatty phase and at least one semi-crystalline copolymer of organic structure having a melting point of greater than or equal to 30° C., obtained from at least two monomers A and B, wherein monomer A is a lipophilic monomer comprising at least one crystallizable side chain and monomer B is a monomer that is copolymerizable with A.

46. The composition according to claim 45, wherein the units derived from monomer A are present in an amount less than 50% by weight of the at least one semi-crystalline copolymer.

47. The composition according to claim 45, wherein monomer B is an amorphous lipophilic monomer.

48. The composition according to claim 45, wherein the at least one semi-crystalline copolymer structures the liquid fatty phase.

49. The composition according to claim 45, wherein the monomer A comprising at least one crystallizable side chain is chosen from at least one monomer of formula X: 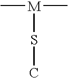
wherein M is an atom of the polymer skeleton,
S is a spacer, and
C is a crystallizable group,
and mixtures thereof, wherein S—C is chosen from alkyl chains comprising at least 11 carbon atoms, which are optionally fluorinated or perfluorinated.

50. The composition according to claim 49, wherein the alkyl chain comprises from 11 to 40 carbon atoms.

51. The composition according to claim 45, wherein the monomer A is a lipophilic monomer comprising vinylic unsaturation comprising an optionally fluorinated hydrocarbon-based crystallizable chain comprising from 11 to 40 carbon atoms.

52. The composition according to claim 45, wherein the monomer A comprising at least one crystallizable side chain is chosen from saturated C₁₄-C₂₄ alkyl (meth)acrylates, C₁₁-C₁₅ perfluoroalkyl (meth)acrylates, C₁₄-C₂₄ N-alkyl-(meth)acrylamides optionally substituted with at least one fluorine atom, vinyl esters comprising C₁₄-C₂₄ alkyl or perfluoroalkyl chains, vinyl ethers comprising C₁₄-C₂₄ alkyl or perfluoroalkyl chains, C₁₄-C₂₄ α-olefins, and para-alkylstyrenes with an alkyl group comprising from 12 to 24 carbon atoms, and mixtures thereof.

53. The composition according to claim 45, wherein the monomer A comprises from 11 to 40 carbon atoms, at least six of which are fluorinated.

54. The composition according to claim 45, wherein the monomer B is a monomer comprising vinylic unsaturation.

55. The composition according to claim 45, wherein the monomer B is a non-ionizable monomer.

56. The composition according to claim 45, wherein the monomer B is a monomer comprising at least one C₄-C₅₀ alkyl group.

57. The composition according to claim 56, wherein the monomer B is a monomer comprising at least one C₈-C₃₀ alkyl group.

58. The composition according to claim 56, wherein the at least one C₄-C₅₀ alkyl group is linear, branched, or cyclic.

59. The composition according to claim 56, wherein the at least one C₄-C₅₀ alkyl group comprises at least one heteroatom.

60. The composition according to claim 59, wherein the at least one heteroatom is chosen from O, N, S, P, and Si.

61. The composition according to claim 56, wherein the hydrogen atoms of the at least one C₄-C₅₀ alkyl group are partially substituted with halogen atoms.

62. The composition according to claim 45, wherein the monomer B comprises at least one branched alkyl group comprises from 4 to 50 carbon atoms.

63. The composition according to claim 62, wherein the at least one branched alkyl group comprises from 8 to 30 carbon atoms.

64. The composition according to claim 62, wherein the at least one branched alkyl group of the monomer B is chosen from isobutyl, tert-butyl, isopentyl, tert-hexyl, 2-ethylhexyl, tert-octyl, isononyl, isodecyl, isododecyl, neodecanoyl, isostearyl, octyldodecyl, methyltetradecyl isomers, methylpentadecyl isomers, methylhexadecyl isomers, dimethyltetradecyl isomers, and dimethylpentadecyl isomers.

65. The composition according to claim 64, wherein the at least one branched alkyl group of the monomer B is chosen from tert-butyl, tert-hexyl, 2-ethylhexyl, tert-octyl, isononyl, isododecyl, neodecanoyl, isostearyl, and octyldodecyl groups.

66. The composition according to claim 45, wherein the monomer B comprises at least one cyclic alkyl group and/or at least one linear alkyl group.

67. The composition according to claim 66, wherein the at least one cyclic alkyl group of the monomer B is chosen from saturated and unsaturated cyclic groups optionally comprising at least one linear or branched C₁-C₁₅ alkyl substituent.

68. The composition according to claim 67, wherein the at least one cyclic alkyl group of the monomer B is chosen from saturated cyclic and cycloalkyl groups.

69. A composition according to claim 68, wherein the at least one cyclic alkyl group of the monomer B is chosen from cyclohexyl, isobornyl, norbornyl, and adamantyl groups.

70. The composition according to claim 69, wherein the at least one cyclic alkyl group of the monomer B is chosen from cyclohexyl and isobornyl groups.

71. A composition according to claim 67, wherein the at least one cyclic alkyl group of the monomer B is chosen from C₄-C₁₅ alkyl benzoate.

72. The composition according to claim 45, wherein the monomer B is chosen from:
alkyl and cycloalkyl (meth)acrylates,
N-alkyl(meth)acrylamides, N,N-dialkyl(meth)acrylamides, and cycloalkyl (meth)
acrylamides,
alkyl vinyl esters and cycloalkyl vinyl esters,
alkyl vinyl ethers and cycloalkyl vinyl ethers,
alkyl allyl ethers and cycloalkyl allyl ethers,
α-olefins comprising an alkyl or cycloalkyl group,
monoalkyl esters, dialkyl esters, and cycloalkyl esters,
maleic acid and itaconic acid amides, and
mixtures thereof.

73. A composition according to claim 72, wherein the monomer B is chosen from maleic acid and itaconic acid esters.

74. The composition according to claim 45, wherein the monomer B comprises at least one linear alkyl group comprising from 4 to 11 carbon atoms.

75. The composition according to claim 74, wherein the monomer B comprises at least one linear alkyl group comprising from 8 to 11 carbon atoms.

76. The composition according to claim 72, wherein the N-alkyl group of the N-alkyl(meth)acrylamides is chosen from n-butyl, n-pentyl, n-hexyl, n-heptyl, n-octyl, n-nonyl, n-decyl, n-undecyl, and mixtures thereof.

77. The composition according to claim 72, wherein at least one of the alkyl groups of the N,N-dialkyl group of the N,N-dialkyl(meth)acrylamides is C₄-C₁₁, and the other at least one alkyl group optionally being identical or different and comprising from 1 to 11 carbon atoms.

78. The composition according to claim 45, wherein the amount of units resulting from the polymerization of the monomer A in the copolymer ranges from 10% to 90% by weight, relative to the weight of the copolymer.

79. The composition according to claim 78, wherein the amount of units resulting from the polymerization of the monomer A in the copolymer ranges from 25% to 75% by weight, relative to the weight of the copolymer.

80. The composition according to claim 45, wherein the amount of units resulting from the polymerization of the monomer B in the copolymer ranges from 10% to 90% by weight, relative to the weight of the copolymer.

81. The composition according to claim 80, wherein the amount of units resulting from the polymerization of the monomer B in the copolymer ranges from 25% to 75% by weight, relative to the weight of the copolymer.

82. The composition according to claim 45, wherein the at least one semi-crystalline copolymer has a weight-average molecular mass ranging from 5,000 to 1,000,000.

83. The composition according to claim 82, wherein the at least one semi-crystalline copolymer has a weight-average molecular mass ranging from 15,000 to 500,000.

84. The composition according to claim 45, wherein the at least one semi-crystalline copolymer has a melting point above the temperature of the keratinous support intended to receive the composition.

85. The composition according to claim 84, wherein the keratinous support intended to receive the composition is chosen from the skin and the lips.

86. The composition according to claim 45, wherein the at least one semi-crystalline copolymer is present in an amount ranging from 0.1% to 80% by weight, relative to the total weight of the composition.

87. The composition according to claim 86, wherein the at least one semi-crystalline copolymer is present in an amount ranging from 5% to 25% by weight, relative to the total weight of the composition.

88. The composition according to claim 45, wherein the at least one semi-crystalline copolymer has a melting point of less than 50° C., and the composition further comprises at least one high-melting crystalline compound having a melting point of at least 50° C.

89. The composition according to claim 88, wherein the at least one high-melting crystalline compound is a polymer having a melting point (m.p.₁) of greater than or equal to 55° C. and less than or equal to 150° C.

90. The composition according to claim 89, wherein the at least one high-melting crystalline compound is a polymer having a melting point (m.p.₁) of greater than or equal to 60° C. and less than or equal to 130° C.

91. The composition according to claim 89, wherein the at least one high-melting crystalline compound is a semi-crystalline copolymer of organic structure, obtained from at least two monomers A and B, wherein monomer A is a lipophilic monomer comprising at least one crystallizable side chain and monomer B is a monomer that is copolymerizable with A, wherein the units derived from monomer A are present in an amount less than 50% by weight of the copolymer.

92. The composition according to claim 89, wherein the at least one high-melting crystalline compound is chosen from:
block copolymers of controlled-crystallization polyolefins,
aliphatic and aromatic polyester polycondensates and aliphatic/aromatic copolyesters,
homopolymers and copolymers comprising at least one crystallizable side chain, and
mixtures thereof.

93. The composition according to claim 88, wherein the at least one semi-crystalline copolymer having a melting point of less than 50° C. and the at least one high-melting crystalline compound having a melting point of at least 50° C. are present in a weight ratio ranging from 90/10 to 10/90.

94. The composition according to claim 93, wherein the at least one semi-crystalline copolymer having a melting point of less than 50° C. and the at least one high-melting crystalline compound having a melting point of at least 50° C. are present in a weight ratio of approximately 50/50.

95. The composition according to claim 45, wherein the composition is in anhydrous form.

96. The composition according to claim 45, wherein the composition is in the form chosen from a paste and a gel.

97. The composition according to claim 45, wherein the composition is in the form chosen from a mascara, an eyeliner, a foundation, a lipstick, a deodorant, a body makeup product, an eyeshadow, a makeup rouge, and a concealer product.

98. A cosmetic makeup process comprising:
applying a composition to keratin materials, wherein the composition comprises: a liquid fatty phase and at least one semi-crystalline copolymer of organic structure having a melting point of greater than or equal to 30° C., obtained from at least two monomers A and B, wherein monomer A is a lipophilic monomer comprising at least one crystallizable side chain and monomer B is a monomer that is copolymerizable with A.

99. A semi-crystalline copolymer of organic structure having a melting point of greater than or equal to 30° C. obtained from at least two monomers A and B, wherein monomer A is a lipophilic monomer comprising at least one crystallizable side chain and monomer B is a monomer that is copolymerizable with A, wherein the units derived from the monomer A are present in an amount less than 50% by weight of the said copolymer.

100. A semi-crystalline copolymer of organic structure having a melting point of greater than or equal to 30° C. obtained from at least two monomers A and B, wherein monomer A is a lipophilic monomer comprising at least one crystallizable side chain and monomer B is an amorphous lipophilic monomer that is copolymerizable with A.
